(19) United States
(12) Patent Application Publication
(10) Pub. No.: US 2025/0244268 A1
(43) Pub. Date: Jul. 31, 2025

(54) Method For Identifying At Least One Component Of A Sample Material

(71) Applicant: Netzsch-Gerätebau GmbH, Selb (DE)

(72) Inventors: Georg Storch, Selb (DE); Natalie Rudolph, Eußenheim (DE); Reinhard Gschwendtner, Markredwitz (DE); Fabian Beckstein, Hof (DE)

(21) Appl. No.: 19/038,195

(22) Filed: Jan. 27, 2025

(30) Foreign Application Priority Data
Jan. 29, 2024 (DE) 10 2024 102 474.7

(51) Int. Cl. G01N 25/18; G01N 33/44

(52) U.S. Cl. CPC G01N 25/18; G01N 33/442

(57) ABSTRACT

A method for identifying at least one component of a sample material, with which a sample is formed. The sample material is formed with a plate-or disk-like geometry or is arranged in a spatial region with a plate-or disk-like geometry. The sample is arranged between two temperature-controllable plate-shaped elements in such a way that the plate-shaped elements extend along a respective one of two opposite main surfaces of the plate-or disk-like geometry. A temperature change of the sample is brought about by bringing about a heat inflow into the sample or a heat outflow out of the sample in a symmetrical manner through the two main surfaces, whereby the temperature change is effected by means of temperature-controlling the plate-shaped elements and covers a temperature range, within which at least one endothermic or exothermic change of the component occurs. A heat flow flowing into the sample or out of it is detected and a profile of the heat flow, which can be represented as curve, as a function of a temperature of the sample or of the plate-shaped elements or of time, is obtained from this; or a temperature of the sample or of the plate-shaped elements is detected, and a profile of a temporal change of the temperature, which can be represented as curve, as a function of temperature or as a function of time, is obtained from this. A curve state of the obtained profile of the heat flow or of the temporal change of the temperature is in each case compared to a reference curve state. The at least one component is identified based on the result of the comparison.

### TECHNICAL FIELD

The invention relates to a method for identifying at least one component of a sample material.

### BACKGROUND

Even though the invention can be useful and can be used in connection with the identification of components in sample materials of a wide variety of types and in a wide variety of fields, the invention and the set of problems, on which it is based, shall be explained in more detail below in an exemplary manner using the example of plastics recycling, but without limiting the invention to this effect.

In the field of the recycling of plastics, it is often the object to determine the composition of the recycled plastic material, for example of a plastic granulate, at different stages of the recycling process, and to receive information about the purity of this material as well as about the extent of potential admixtures. In particular, proportions of unwanted polymer types, which may be present in the analyzed material, in particular recyclate, are to be detected.

Widespread methods in the field of plastics recycling, which are rather intended for the process of sorting the starting material, are the IR spectroscopy, the utilization of cameras in connection with image recognition, further optical methods or a separation of the materials according to their density. None of these conventional methods, however, can cover the entire field of relevant plastics. For example, the IR spectroscopy is thus not sensitive to black plastics. In particular, the above-mentioned methods additionally fail when analyzing components in compounded substance mixtures, for instance for the purpose of quality control.

It is known, for example, to determine melt or glass transition temperatures, in particular of plastics, by means of the method of the so-called differential scanning calorimetry (DSC in short). It is possible hereby to draw a conclusion to the type of the analyzed plastic on the basis of peaks of the measured variable obtained by means of the DSC method. The method of the heat flow differential scanning calorimetry is generally able to recognize many plastics and provides a high sensitivity.

However, large material quantities are processed in the plastics recycling industry, which are to be checked for the purity and composition thereof. However, the material samples analyzed in a conventional manner by means of differential scanning calorimetry and the devices used for this purpose, are very small, the quantities thereof lie in the range of milligrams. The analysis of an individual sample from a large material batch, for instance from a silo or the like filled with plastic granulate, by means of DSC, therefore does not make it possible to obtain a reliable, representative image of the entire batch. An improvement of the information by analyzing a plurality of samples from the batch by means of DSC is extremely complex and time-consuming

An apparatus for performing a STA (simultaneous thermal analysis) for larger weighted samples is described in the article “Simultaneous thermal analysis of large samples” by G. Matuschek et al., Journal of Thermal Analysis, Vol. 47 (1996), pages 623 et seqq. With regard to a use in the recycling industry, however, the acquisition of this instrument is relatively expensive, and the application is relatively complicated. In addition, the sample distribution can be unfavorable. A more cost-efficient approach, which is easier to handle, would be desirable for the use in the recycling sector.

Apparatuses, which are referred to as HFM equipment, are further known, whereby HFM stands for the English expression “heat flow meter”. The heat transfer through a sample is conventionally measured by means of such an apparatus, for example in order to determine the insulating properties of an insulating jacket. For this purpose, the sample is arranged between two temperature-controllable plates in the HFM apparatus, a temperature gradient is applied to the sample with the help of the plates and, in the stationary state after reaching a thermal balance, the heat flow flowing through the sample is measured and the heat transfer is measured numerically therewith.

It is likewise known to measure the specific heat capacity Cp, for example of a polymer or of an insulating material, by means of an HFM apparatus. For this purpose, both plates are initially held at the same temperature and are then subjected to a short temperature ramp, again followed by isothermal conditions. The integral of the heat flow is evaluated hereby.

A mode of operation of an HFM apparatus, in the case of which both plates are brought to the same temperature, whereby a gradual isothermal temperature program serves the purpose of determining the heat storage capacity of a sample, in terms of sensitive and latent heat, is furthermore described in the technical standard ASTM C1784.

The WO 2022/250533 A1 deals with a method of differential scanning calorimetry as well as a device for a differential scanning calorimetry, whereby the determination of thermodynamic properties of a material as well as of the composition thereof are described. In the case of the approach described in the WO 2022/250533 A1, the plate-shaped sample is heated or cooled on one side. A plurality of temperature sensors arranged so as to be distributed in a planar manner in a grid, is provided on the opposite side of the sample.

In light of the foregoing, it would be desirable to be able to correctly characterize a larger batch of a material, for example in the field of plastics recycling, in a simple, easy to handle as well as time-and cost-efficient manner and to be able to get an exact idea about the composition thereof and possible contaminations hereby.

### SUMMARY

In light of the foregoing, it is an object of the invention to specify an efficient and reliable method, by means of which a sufficiently large sample quantity can be analyzed, in order to characterize a material batch as representatively as possible. The method is to in particular be suitable and functional for correctly characterizing material batches at industrial scales by means taking and analyzing samples.

According to the invention, this object is solved by means of a method with the features of claim 1.

According to this, a method for identifying at least one component of a sample material is proposed, with which a sample is formed, whereby the method comprises:
providing the sample, whereby the sample material is formed with a plate-or disk-like geometry or the sample material is arranged in a spatial region with a plate-or disk-like geometry;
arranging the sample between two temperature-controllable plate-shaped elements in such a way that the temperature-controllable plate-shaped elements extend along a respective one of two opposite main surfaces of the plate-or disk-like geometry;
bringing about a temperature change of the sample by bringing about a heat inflow into the sample or a heat outflow out of the sample in a symmetrical manner through the two main surfaces of the plate- or disk-like geometry, whereby the temperature change is effected by means of temperature-controlling the plate-shaped elements and covers a temperature range, within which at least one endothermic or exothermic change of the at least one component of the sample material occurs;
detecting a heat flow flowing into the sample or out of the sample and obtaining from this a profile of the heat flow, which can be represented as curve, as a function of a temperature of the sample or of the temperature-controllable plate-shaped elements or as a function of time, or
detecting a temperature of the sample or of the temperature-controllable plate-shaped elements, and obtaining from this a profile of a temporal change of the temperature, which can be represented as curve, as a function of temperature or as a function of time;
comparing a curve state, in particular including a curve shape, of the obtained profile of the heat flow or of the temporal change of the temperature over temperature or time in each case to a reference curve state of one or several reference profiles; and
identifying the at least one component of the sample material based on the result of the comparison.

An idea, on which the invention is based, is to generally analyze the sample material based on the method of the differential scanning calorimetry (DSC), but on a sample, which includes a much larger material quantity than in the case of the DSC. It can be accomplished in this way to representatively depict an inhomogeneous composition of the material to be analyzed, for example recyclate, in a significantly improved manner by means of the sample and to correctly identify the component(s) of a mixture.

In the case of the invention, an HFM arrangement, which is known as such, can be used in a DSC-similar manner for this purpose. The HFM apparatus can hereby correspond to an apparatus, as it is mentioned, for example, in the technical standard ASTM C1784 and references cited therein, or can be formed similar thereto. Depending on the sample materials to be analyzed, however, the HFM apparatus can be modified, for example with regard to the temperature range, which can be covered. It may thus be expedient for the advantageous application to the field of plastics recycling when the HFM apparatus provides for a larger temperature range and higher maximum temperatures by means of modified temperature-control units.

Advantageously, the heat loss to the outside can be reduced and well-defined temperature gradients and a heat flow, which is one-dimensional at least with a very good approximation, can be achieved by means of the method according to the invention by means of the essentially symmetrical introduction or removal of heat through the two main surfaces, in other words the symmetrical application of the sample with a heat flow or the symmetrical impressing of a temperature change, in connection with the plate-or disk-shaped geometry of the sample material or of the region, in which it is arranged, respectively. This contributes in an advantageous manner to exact, reliable measuring results and to a reliable identification of the component(s).

The plate-or disk-shaped geometry of the sample material is additionally advantageous to that effect that the endothermic/exothermic process in the sample material, which is induced by means of the heat supply or removal, for instance a melt or glass transition of a component thereof, takes place quickly and no significant temperature gradients and thus also no “melt front” can thus develop within the sample. This is achieved in that a large heat input surface is made possible by means of the provided geometry in spite of a relatively large, weighted sample.

In the case of the present invention, the identification of the component(s) can additionally be accomplished in an advantageous manner, without a simultaneous measurement on a reference material or a simultaneous empty measurement becoming necessary as reference. This contributes to a reduction of the amount of equipment and the costs associated therewith as well as to a simplification when carrying out the method. The identification of the component(s) is thus not only reliable but also saves time and is possible easily and is thus advantageous for the use, for example, in the field of plastics recycling, or other processes, at an industrial scale.

Advantageous designs and further developments of the invention follow from the subclaims as well as from the description with reference to the drawings.

In one design, the sample material is subjected to a continuous temperature profile, in particular a predefined temperature ramp, preferably a temperature ramp, which rises or falls linearly over time, when bringing about the temperature change of the sample.

In a further development, the bringing about of the temperature change of the sample takes place in such a way that a heat flow, which is constant over time, is supplied to the sample or is removed from the sample.

According to one design, the at least one component of the sample material runs through a phase transition and/or a glass transition within the temperature range covered during the temperature change. Changes of this type of the components of the sample material with the temperature thereof can be used well for identifying one or several components.

In a further development, the sample material is melted or frozen completely or partly during the temperature change. Melting or freezing as phase transitions between solid and liquid phase are likewise useful to characterize the component(s) of the sample material.

The temperature-controllable plate-shaped elements in particular serve the purpose of heating and/or cooling the sample.

In the case of one design, the sample material is subjected to two or more temperature change processes, in particular to one or several heating processes and one or several cooling processes, by means of temperature-controlling the temperature-controllable plate-shaped elements. This can be used for an even better identification of the component(s). Depending on the sample material or the components thereof, respectively, the temperature-dependent processes in the sample material can be influenced with the help of defined heat-up and/or cool-down rates. A second heat-up and the evaluation thereof can in particular be used for establishing a uniform initial state, for example by means of preceding melting, and for carrying out the evaluation based on a defined initial state of this type. In particular the second heat-up as well as further a subsequent cool-down phase, during which, for example, a crystallization can be observed, can be of interest and can be used for the comparison of the curve state to the reference curve state. A cool-down phase of this type can make it possible, for example, to draw conclusions to additives, which may be contained in the sample, as crystallization accelerator.

According to one design, a heat flow, which in each case flows from the temperature-controllable plate-shaped element into the sample or vice versa, is in each case detected between the temperature-controllable plate-shaped element and the side of the sample facing the temperature-controllable plate-shaped element in the region of the main surface of the plate-or disk-like geometry, to which the temperature-controllable plate-shaped element is adjacent, in particular in each case by means of a heat flux transducer, for example of a heat flux transducer, which is formed in a planar manner. This provides for a reliable, exact and direct detection of the heat flow. The two-sided detection of the heat flow can also be used, for example, for controlling the symmetry of the mode of operation.

According to one design, the heat flux transducers are in each case arranged in a planar manner in a central region of one of the main surfaces of the plate-or disk-like geometry between the temperature-controllable plate-shaped element adjacent to this main surface and the sample. The heat flux transducer can in particular in each case assume a large central region of the main surface. In the case of some exemplary designs, the central region of the main surface occupied by the heat flux transducer can, viewed in the main surface, have a size of at least one third or of at least half of the dimension of the main surface in each of two, in particular orthogonal, coordinate directions. In some exemplary designs, the central region occupied by the heat flux transducer as measuring surface can in particular occupy at least one ninth or at least one sixth or at least one fourth of the surface area of the main surface. It can be achieved in this way that an influence of boundary effects, which can hardly be avoided entirely, is negligibly small or does not exist due to the finite sizes of the sample and of the temperature-controllable plate-shaped elements. A planar expansion of the heat flux transducer in the mentioned large central region makes it possible to include a large portion of the sample material for good information about it. A further increase of the proportion of the main surface occupied by the heat flux transducer as active measuring surface beyond the above-mentioned values can be advantageous, in order to achieve an even better averaging over the possibly inhomogeneous sample.

In a further development, one or both of the temperature-controllable plate-shaped elements is/are moved towards the sample prior to bringing about the temperature change of the sample, whereby, conveyed via the temperature-controllable plate-shaped elements, a predefined force is applied to the sample in a thickness direction of the plate-or disk-like geometry or a predefined thickness of the sample is adjusted in the thickness direction of the plate-or disk-like geometry. Defined conditions, such as, for example, a defined thickness of the sample can be created in this way for the analysis to be performed, a reliable contact with the sample material for a defined temperature control can be achieved and a heat transfer resistance, which is as small and even as possible, can be ensured at the contact surface.

In one design, it is in particular provided that the sample material is provided with a casing, which encloses the sample material in a closed sample volume. Alternatively, it can in particular be provided in a different design that the sample material is surrounded circumferentially on the edge side by a flexible sealing element, which extends in the thickness direction of the sample essentially over the thickness or filling height of the sample and which, together with the temperature-controllable plate-shaped elements, is formed for enclosing the closed sample volume, whereby the sealing element is in particular configured for coming into a sealing contact with the temperature-controllable plate-shaped elements in each case. In that the casing or the sealing element is provided as a type of edge, a flow-off of the sample material, which liquefies during a melting, can be prevented. By means of its flexibility, the sealing element preferably also provides for a tracing by moving the plate-shaped element(s), in order to maintain a contact of the sample material with the plate-shaped elements during the melting.

According to one design, it is provided that the temperature of the sample is detected on a surface of the sample or in an interior of the sample and/or that the temperature of the temperature-controllable plate-shaped element is in each case detected on a surface thereof or in an interior thereof.

In the case of a further development, the comparing of the curve state and of the reference curve state is carried out by using corresponding abscissa and/or ordinate values, which in each case characterizes the curve shape and the reference curve shape at least in sections.

In the case of another further development, the comparing of the curve state and of the reference curve state is carried out by means of at least one characteristic level and/or of at least one characteristic extremum, in particular peak, in each case of the obtained profile of the heat flow or of the temporal change of the temperature as well as of the reference profile, whereby the level or the extremum in each case corresponds in particular to a conversion within the sample material.

In one design, at least one abscissa value, which corresponds to an onset point or endpoint or midpoint or turning point or extreme value assigned to the level or the extremum within the curve shape, is determined for at least one level or at least one extremum, in particular a peak, of the obtained profile of the heat flow or the temporal change of the temperature, and is compared to at least one corresponding abscissa value of at least one reference profile.

According to a further design, it is provided that a height of the level is determined for at least one level of the obtained profile of the heat flow or of the temporal change of the temperature, and is compared to at least one height of at least one level of at least one reference profile, and/or that the assigned extreme value of the heat flow or of the temporal change of the temperature is determined for at least one extremum, in particular a peak, of the obtained profile of the heat flow or of the temporal change of the temperature, and is compared to an extreme value of at least one extremum, in particular peak, of at least one refence profile.

An evaluation of the curve state according to the above explanations and further developments, which can be combined for the further improvement, provides for a reliable identification of the component(s).

In the case of a further development, at least one characteristic integral value, in particular a surface below at least one section of the obtained profile of the heat flow represented as curve or of the temporal change of the temperature, for example under a peak, is determined and compared to a characteristic integral value of the reference profile. In the case of this further development, the identification of the at least one component of the sample material is additionally carried out based on the result of the comparison of the characteristic integral values. This can contribute to a further improvement of the identification. The integral value can be equivalent to an enthalpy difference, for instance in the case of a phase transition in the sample, or can correspond to a difference in the specific heat capacity, for instance in the case of a glass transition in the sample.

In a further design, the obtained profile of the heat flow or the temporal change of the temperature is compared directly to the reference profile, in each case in the entirety thereof.

In the case of a further development, the comparing of the curve state, in particular curve shape, and of the reference curve state, in particular reference curve shape, comprises the use of a pattern recognition algorithm, for example of an adaptive algorithm and/or of an artificial intelligence method.

The accuracy and reliability of the identification can be advanced even further by means of a direct comparison and/or use of pattern recognition or artificial intelligence.

According to one design, a plurality of profiles of the heat flow or of the temporal change of the temperature, which are in each case determined beforehand for a known sample material or sample material mixture, are used as reference profiles.

According to one design, the reference profiles are provided in the form of a data collection, which includes reference profiles of an application-specific selection of pure substances and mixtures, for example plastics and plastic mixtures.

In that a plurality of reference profiles can be resorted to, for example on the basis of the data collection, which can be provided, for example, in the form of a database, it becomes possible to provide reliable information about a wide range of possible admixtures or contaminations of a sample. A method of this type can turn out to be very useful, for example, for controlling recycling processes, in the case of which, by nature, hardly a complete certainty can be obtained about the starting materials.

The data collection, for instance database, can be provided, for example, on a suitable storage medium or can be accessible via a network.

In the case of a further development, the sample material is provided as a loose material with a plurality of individual pieces, for example as a granulate or a powder.

In the case of another further development, the sample material is provided as a cohesive body.

A provision as loose material or solid body provides for the use in the case of various types of samples.

According to one design, the detection of the profile of the heat flow or of the temperature takes place in the absence of a simultaneous reference measurement, in particular on a reference, which geometrically corresponds to the sample and which is formed with a previously known material, or by means of empty measurement as reference, within the same analysis arrangement or by means of a further identical analysis arrangement. The amount of equipment as well as the handling expenditure during the analysis of the sample are significantly reduced in this way, which contributes to a reduction of costs and expenditure of time. An empty “twin” of the analysis arrangement, for example as reference, is not required. A simple, goal-oriented analysis of the sample material by avoiding possible error sources thus becomes possible.

In one design, the analyzed sample has a mass of more than approximately 10 grams, for example between approximately 10 grams and approximately 10 kilograms, preferably between approximately 10 grams and approximately 1 kilogram, more preferably between approximately 10 grams and approximately 200 grams, for example between approximately 10 grams and approximately 100 grams. Samples of this type make it possible to get a reliable and representative idea about a larger material batch, as it is found at the industrial scale, for example in the recycling sector.

According to a further development, it is provided that the sample material is a mixture and that a material or several materials, which is/are contained in the sample, is/are identified by means of the method.

In an advantageous design, the method is used for determining the purity of a plastics recyclate, in particular for identifying possible admixtures or contaminations and/or for determining an extent thereof.

The method can in particular be used in a process chain during the plastics recycling. A control of the recycling process can thus be made possible in a time-saving and simple manner at different stages thereof, as well as a quality control of the obtained end product, for instance of a recycling plastic granulate, can be made possible.

In an exemplary further development, a compounded substance mixture, in particular a compounded plastic, is provided as the sample material of the sample. The invention makes it possible in an advantageous manner to also analyze a plastic, which has already been processed in this way, with regard to its components.

### BRIEF DESCRIPTION OF THE DRAWINGS

The invention will be described in more detail with reference to exemplary embodiments, which are illustrated in the enclosed drawings.

The enclosed drawings are included, in order to provide a further understanding of this invention and are included in this description and represent a part thereof. The drawings illustrate the embodiments of this invention and, together with the description, serve the purpose of explaining the principles of the invention. Other embodiments of this invention and many of the provided advantages of this invention can be understood easily when they become easier to understand by means of a reference to the following detailed description. The elements of the drawings are not necessarily drawn at the same scale to one another. Identical reference numerals accordingly identify similar parts.

FIG. 1 shows a schematic cross-sectional view of an arrangement, which is used for carrying out a method according to exemplary embodiments of the invention;

FIG. 2 shows, in perspective and schematically, a plate-or disk-like geometry of the sample material, which is used for carrying out the method according to the exemplary embodiments, or of a spatial region, in which the sample material is arranged for carrying out the method according to the exemplary embodiments;

FIG. 3 shows an exemplary measuring result, which is obtained by means of the arrangement illustrated schematically in FIG. 1 and which, according to an exemplary embodiment, is used for identifying at least one component of a sample material, measured, for example, for a sample of a polylactide (PLA);

FIG. 4 shows an exemplary measuring result for a polylactide (PLA), which was obtained by means of a conventional method of the differential scanning calorimetry (DSC), whereby two heat-ups are shown in an exemplary manner;

FIG. 5 shows an exemplary measuring result, which was obtained by means of the arrangement illustrated schematically in FIG. 1 and which, according to a further exemplary embodiment, is used for identifying at least one component of a sample material, for example measured for a sample of a paraffin;

FIG. 6 shows an exemplary measuring result for a paraffin, which was obtained by means of a conventional method of the differential scanning calorimetry (DSC);

FIG. 7 shows a schematic flowchart of a method according to exemplary embodiments of the invention;

FIG. 8 schematically illustrates an exemplary casing for the sample material for carrying out methods according to exemplary embodiments of the invention; and

FIG. 9 schematically illustrates an edge-side sealing element for enclosing the sample material, for carrying out methods according to further exemplary embodiments.

Identical reference numerals in the figures refer to identical or functionally similar component parts, unless otherwise specified. All directional terms, such as “top”, “bottom”, “left”, “right”, “above”, “below”, “horizontal”, “vertical”, “back”, “front” and similar terms are used only for explanatory purposes and shall not limit the embodiments to the specific arrangements, which are illustrated in the drawings.

### DETAILED DESCRIPTION

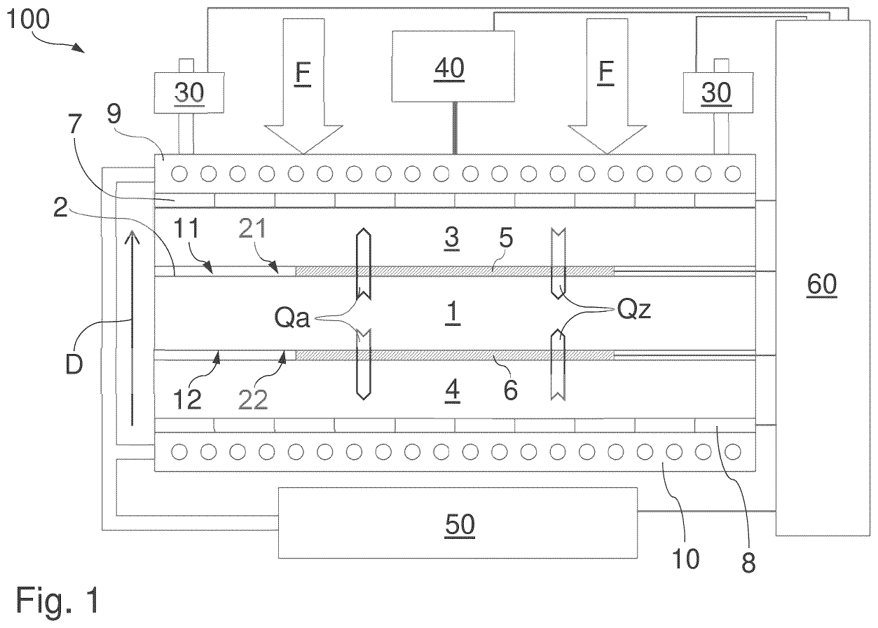

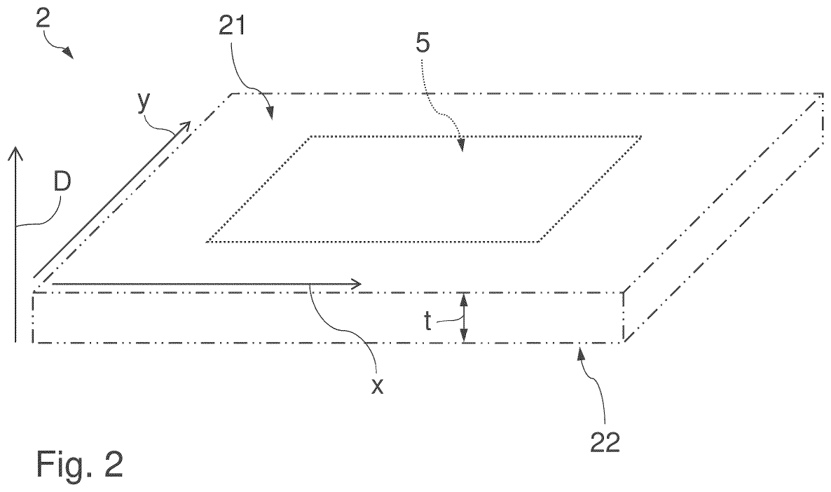

FIG. 1 shows an arrangement 100, which can be used for carrying out methods according to below-explained exemplary embodiments of the invention. The arrangement 100 serves the purpose of analyzing a sample 1. To provide the sample 1, a sample material, which is taken, for example, from a batch of a loose plastic granulate obtained from a recycling process, is arranged in a spatial region with a plate-or disk-like geometry 2. For this purpose, the sample material can be filled into a sample frame, which is not illustrated in more detail, which defines the geometry 2. Alternatively, the sample material of the sample 1 can be a cohesive solid body or alternatively a compressible body, which each has the plate-or disk-like geometry 2, for example, is custom-tailored. In an exemplary manner, FIG. 2 shows a plate-or disk-shaped geometry 2 of this type in a schematic manner. The geometry 2 is formed to be flat and in the shape of a cuboid in FIG. 2, whereby extensions of the geometry 2 in the two coordinate directions x and y, which are orthogonal to one another, are significantly larger than an extension or thickness t of the geometry 2 in the thickness direction D perpendicular to x and y. Main surfaces 21 and 22 of the geometry 2 are essentially of identical shape and size and are formed in parallel, and are rectangular, for example, in the case of the shown exemplary embodiment in FIG. 2.

The sample material of the sample 1, regardless of the physical form thereof, for instance as granulate or cohesive body or otherwise, is in particular a mixture, with at least one main material, to which one or more other materials can be admixed due to the recycling process, in particular as contamination of the main material, which is desired to be as pure as possible. The sample material thus comprises components, whereby this term refers to the main component(s) as well as to possible undesired admixtures.

In the case of the arrangement 100 of FIG. 1, the sample 1 is arranged in a sandwich-like manner between two temperature-controllable plate-shaped elements 3 and 4. The arrangement 100 further has a heat flux transducer 5 arranged between the main surface 21 of the geometry 2 and the plate-shaped element 3, and a heat flux transducer 6 arranged between the main surface 22 of the geometry 2 and the plate-shaped element 4.

In the case of the arrangement 100 of FIG. 1, a heat flow, which flows through the main surfaces 21, 22 from the temperature-controllable element 3 or 4, respectively, into the sample material or out of the latter, respectively, into the element 3 or 4, respectively, can be measured on both temperature-controlled sides 11, 12 of the sample 1 by means of the heat flux transducers 5 and 6. The side 11 of the sample 1 hereby faces the temperature-controllable element 3, the side 12 facing the temperature-controllable element 4. The heat flux transducer 5, 6 is formed for detecting a heat flow flowing through it. For example, the heat flux transducer 5, 6 can provide an output signal, which is proportional to the heat flow, which flows through, and from which a numerical absolute value for the heat flow can be obtained after the calibration.

The heat flux transducers 5 and 6 are each formed in a planar manner and are each arranged in a central region of the assigned main surface 21 or 22. In an exemplary manner, an outline of the heat flux transducer 5 is suggested schematically in FIG. 2 in a dotted line, whereby the heat flux transducer 6 is arranged analogously and symmetrically to the transducer 5 in the region of the opposite main surface 22.

Each of the heat flux transducers 5, 6 occupies a comparatively large central region of the main surface 21 or 22, respectively, which, viewed for example in the main surface 21 or 22, has a size of at least one third of the dimension of the main surface 21 or 22 in each of the coordinate directions x and y. This helps to minimize the influence of boundary effects and to simultaneously detect the heat flow through a large region of the sample material. In FIG. 2, for example, each heat flux transducer 5, 6 occupies a central region as an active measuring surface, which corresponds approximately to one fourth of the surface area of the main surface 21 or 22. Other, in particular larger surface portions, however, are conceivable for the further improvement of the averaging over the sample 1.

The arrangement 100 further comprises a heat sink 9, which is assigned to the temperature-controllable plate-shaped element 3 and which extends parallel to the element 3, as well as a heat sink 10, which is assigned to the temperature-controllable plate-shaped element 4 and which extends parallel to the element 4. The cooling of the heat sinks 9, 10 itself takes place by means of a cooling system 50.

A temperature-control system 7 or 8, respectively, for the temperature control is furthermore in each case arranged between the heat sink 9 and the element 3 as well as the heat sink 10 and the element 4. For example, the temperature-control system 7, 8 can in each case be formed as a or with a heating unit, such as, for instance, a resistance heating unit. For example, a temperature range, which is advantageous when the invention is applied to the field of plastics recycling and which can include, for example, temperatures of 200 degrees Celsius or more, can be covered well with a heating unit of this type. In the case of other applications of the invention, in which lower temperatures are used, the temperature-control system 7, 8 can in each case alternatively be formed as a Peltier system. A Peltier system can provide the advantage that it provides a heating as well as a cooling.

An assembly formed with the heat sink 9, the temperature-control system 7 and the plate-shaped element 3 can additionally be displaced essentially perpendicular to a main extension plane of the sample 1 and thus perpendicular to the main surfaces 21, 22 by means of displacement units 30. A force F in the thickness direction D can be applied to the sample 1 by means of the units 30. A displacement path or a thickness t of the sample 1 can be detected in the thickness direction D of the sample 1 by means of a thickness measuring unit 40. In one variation, both elements 3 and 4, in addition to respectively assigned heat sink 9, 10 and temperature-control system 7, 8, can be capable of being displaced one on top of the other and towards the sample 1.

The temperature-control systems 7 and 8, the cooling system 50, the displacement units 30, the thickness measuring unit 40 and the heat flux transducers 5, 6 as well as optionally additional temperature sensors, for instance thermocouples, which can be provided at the points, which will be described in more detail below, in different combination, are coupled to a data processing and control system 60, which makes it possible to control in particular the units and systems 30, 50, 7, 8, and to process measured values detected by means of the unit 40 and the heat flux transducers 5, 6 as well as the mentioned temperature sensors. The system 60 can additionally be coupled to non-illustrated input and output units, storages as well as further units. It is also conceivable to couple the system 60 to a network interface.

Even though the main surfaces 21, 22 and thus the main extension planes of the sample 1 as well as of the elements 3, 4 are aligned horizontally in an exemplary manner in the case of the exemplary embodiment of FIG. 1, a vertical alignment or any other alignment in space are likewise conceivable.

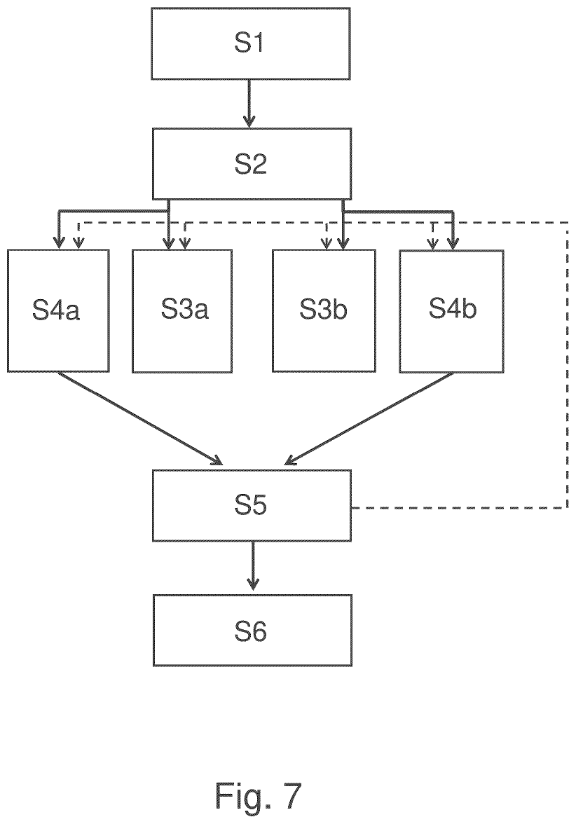

Methods according to exemplary embodiments of the invention will be explained below, based on a schematic illustration of the process sequence given in FIG. 7, and exemplary results, which are obtained by means of methods of this type, and, for comparison, results of conventional DSC measurements will further be discussed.

FIG. 7 shows a method for identifying at least one component of the sample material, with which the sample 1 is formed.

In a first step S1 of the method, the sample 1 is provided, as described above, and the sample material is hereby arranged in a spatial region with the plate-or disk-like geometry 2, see FIG. 2, for example as a loose material, for instance the above-mentioned granulate. Alternatively, the sample material of the sample 1 is formed with the plate-or disk-like geometry 2 in step S1, for example as compact solid body or as a compressible body. A mass of the sample 1 is larger than approximately 10 grams and lies, for example, in a range of between approximately 10 grams and approximately 10 kilograms, preferably between approximately 10 grams and approximately 1 kilogram, more preferably between approximately 10 grams and approximately 200 grams. A further preferred mass of the sample 1 lies, for example, between approximately 10 grams and approximately 100 grams. Depending on the mass of the sample 1, the dimensioning can be adapted so as to correspond to the arrangement 100, for instance with respect to the dimensions, the mechanical stability and the heating and cooling capacity.

In a second step S2, the sample 1 is arranged between the two temperature-controllable plate-shaped elements 3 and 4. This is carried out in such a way that the temperature-controllable plate-shaped elements 3 and 4 extend along a respective one of the two opposite, essentially flat main surfaces 21 and 22 of the geometry 2. The sample 1 and the plate-shaped elements 3 and 4 are thus arranged parallel to one another in an essentially sandwich-like manner. The planar heat flux transducer 5 is hereby arranged between the main surface 21 and the element 3, and the planar heat flux transducer 6 is arranged between the main surface 22 and the element 4. The heat flux transducers 5 and 6 are preferably each fixedly integrated into the plate-shaped element 3 or 4 and are brought into contact with the main surfaces 21, 22, after the sample 1 was brought between the elements 3, 4.

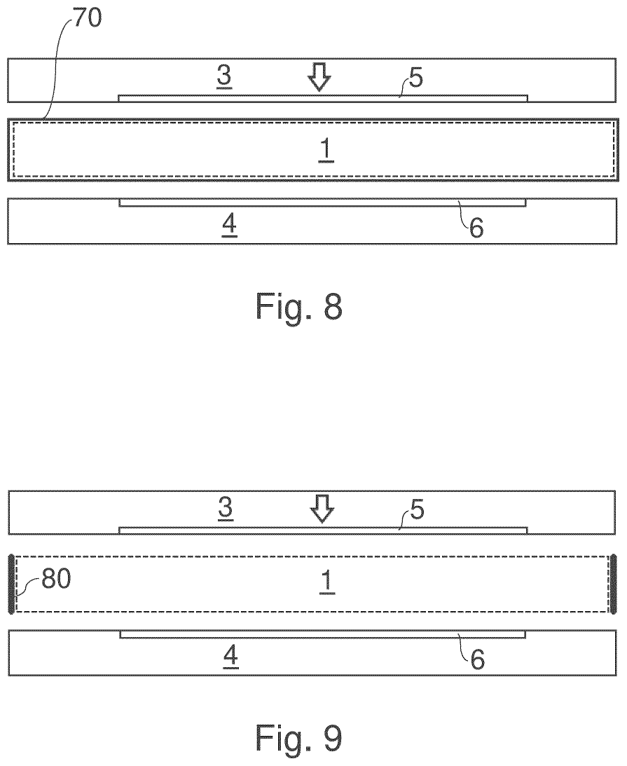

So that the sample material does not leak during a subsequent heat-up and melting or liquefying, at a phase or glass transition, the sample material of the sample 1 is surrounded with a casing 70 or a flexible sealing element 80 in exemplary embodiments, see FIGS. 8 and 9, in which the casing 70 or the sealing element 80, respectively, together with the elements 3, 4, which are still spaced apart from the sample 1, are shown for the schematic illustration. The casing 70 or the sealing element 80 can alternatively be used in the arrangement 100 of FIG. 1, in order to carry out the methods of the exemplary embodiments described here, but are not shown in FIG. 1 to create a better overview.

The casing 70 in FIG. 8 surrounds the sample material of the sample 1 completely and encapsulates it into a closed volume. The casing 70 can be formed with a flexible material. In contrast, the sealing element 80 of FIG. 9 surrounds the sample material of the sample 1 on the edge side and in a frame-like manner along the circumference of the sample 1, whereby the sealing element 80 can come into contact with each of the temperature-controllable plate-shaped elements 3, 4 so as to form a seal.

In step S2, after arranging the sample 1 between the elements 3, 4 in the manner described just now, the upper configuration in FIG. 1 with the plate-shaped element 3 as well as the temperature-control system 7 assigned thereto and the heat sink 9, is moved towards the sample 1 by means of the displacement units 30. A defined contact with low, even heat transfer resistance can be achieved hereby on the main surfaces 21, 22 and, in the case of compressible and/or loose materials, a defined thickness t of the sample material. A defined adjustment and control of the thickness t is made possible by means of the thickness measuring unit 40. The displacement units 30 are preferably controlled hereby by the data processing and control system 60, which is additionally connected to the measuring unit 40 and which records the detected distance or thickness values thereof and considers them during the control of the displacement units 30 and can display these values and/or store them for documentation purposes. A predefined force or load F can hereby be applied to the sample 1 by means of the displacement units 30. The load F can be detected, for example, by means of a force measuring unit, which is not illustrated in FIG. 1, and can be adjusted to a target value. If the sealing element 80 is used, the method described just now takes place by means of the displacement units 30 in such a way that the sealing element 80 comes to rest against both elements 3 and 4 so as to form a seal.

In a third step S3a or S3b, a temperature change of the sample 1 is brought about. For this purpose, a heat inflow Qz into the sample 1 or a heat outflow Qa out of the sample 1 through the two main surfaces 21 and 22 of the plate-or disk-like geometry 2 is brought about in a symmetrical manner. Due to the symmetrical mode of operation of the arrangement 100, essentially half of the total heat flow can thus in each case flow to or flow off via one of the temperature-controllable plate-shaped elements 3 and 4, in the context of the accuracy, which can be achieved with the components of the arrangement 100 and in the case of sufficient homogeneity of the sample 1.

The temperature change of the sample 1 is effected by means of temperature-controlling the plate-shaped elements 3, 4 by means of temperature-controlling the plate-shaped elements 3, 4 with the help of the respectively provided temperature-control system 7, 8 and the respectively provided heat sink 9, 10. For this purpose, the temperature-control system 7, 8 and the cooling system 50 connected to the heat sinks 9, 10 is controlled by means of the data processing and control system 60.

The temperature change effected in the third step S3a or S3b covers a temperature range, within which at least one endothermic or exothermic change of the at least one component of the sample material occurs. A change of this type is preferably a phase transition, for example a melting or freezing of at least one component or of several components of the sample material, or a glass transition of one or of several such component(s), or a phase as well as a glass transition, provided that one component has, for example, crystalline and amorphous proportions.

According to alternative exemplary embodiments of the invention, two different ways are provided in order to bring about the temperature change. This is made clear in FIG. 7 by means of two alternative steps S3a or S3b.

According to step S3a, the temperature change is carried out in such a way that the sample material is subjected to a predefined temperature profile, which is stored, for example, in a non-illustrated storage unit, which the data processing and control system 60 can access. This temperature profile is selected continuously and in particular as a predefined temperature ramp, which rises or falls linearly over time, and which covers the above-mentioned temperature range. The temperature is hereby detected on a main surface 21, 22 of the sample 1 or both main surfaces 21, 22, for example, by means of a temperature sensor or temperature sensors, respectively, or the temperature is detected in the interior of the sample 1. Alternatively, or additionally, a detection of the temperature on a surface facing the main surface 21 or 22, respectively, of the plate-shaped element 3 or 4 or of both or in the interior of one or of both plate-shaped elements 3, 4 is possible by means of temperature sensor(s). The temperature profile to be prescribed can correspond to the temperature at one of those points or can correspond to a temperature, which can be modeled, for example, based on the temperature(s) at one or several of the mentioned temperature measuring points. In step S3a, the temperature change is impressed symmetrically from both sides 11, 12 of the sample 1. In the case of a symmetrical temperature control of this type of the elements 3, 4 and thus symmetrical mode of operation and symmetrical bringing about of a temperature change of the sample 1, a difference in the heat flow, which is in each case supplied to or removed via the element 3 and the element 4, can result in the case of pronounced inhomogeneity of the sample 1 due to different heat capacities or heat conductivities of components of the sample material.

According to the alternative step S3b, the temperature change of the sample is brought about in such a way that a heat flow, which is constant over time, is supplied to the sample 1 or is removed from the sample 1. This is reached symmetrically by temperature-controlling the temperature-controllable, plate-shaped elements 3 and 4, whereby the entered or removed heat flow is controlled or preferably regulated to a predefined constant value by means of the data processing and control system 60. In particular, the heat flow supplied or removed in each case by means of the two elements 3 and 4 is regulated in step S3b to the same value for a symmetrical operation. For this purpose, the heat flow, which is actually present, is measured with the help of the heat flux transducers 5, 6. An additional measurement of the temperature of the sample 1 on the main surfaces 21, 22 and/or in the interior of the sample 1, as well as alternatively or additionally on surfaces of the plate-shaped elements 3, 4 and/or in each case in the interior thereof, as described above for step S3a, is conceivable for the improvement of the regulation of the heat flow.

A readjustment can take place by means of the displacement units 30 during the temperature changes of the sample 1 according to step S3a or S3b, so that a good contact to the sample 1 and a good heat transfer to/from the plate-shaped elements 3 and 4 is maintained, for example in the case of a phase or glass transition with liquification.

While the temperature change of the sample 1 is carried out according to step S3a or S3b, a heat flow or a temperature is detected simultaneously in a fourth step S4a or alternatively S4b.

In detail, a heat flow flowing into the sample 1 or out of the sample 1 is detected by means of the heat flux transducers 5, 6 in step S4a, while the sample 1 is subjected to the defined, impressed temperature profile. A profile of the heat flow \dot{Q}(T) or \dot{Q}(t), which can be represented as curve, is obtained from the detected heat flow values as a function of the temperature T of the sample 1 or of the temperature-controllable plate-shaped elements 3, 4 or instead as a function of time t.

Alternatively, a temperature of the sample 1 or of the temperature-controllable, plate-shaped elements 3, 4 is detected in step S4b, while the defined, constant heat flow is impressed on the sample 1 and is thus introduced into the sample 1 or is removed therefrom. A profile of a temporal change of the temperature dT/dt (t) or dT/dt (T), which can be represented as curve, is obtained from the detected temperature values as a function of temperature or as a function of time.

For step S4a, S4b, the temperature of the sample 1 or of the plate-shaped elements 3, 4 can be detected at the points described above with regard to step S3a and S3b.

In a fifth step S5, the curve state of the obtained profile, which can be represented as curve, either of the heat flow \dot{Q}(T) or \dot{Q}(t) or of the temporal temperature change dT/dt (t) or dT/dt (T) is compared to a reference curve state of a reference curve, preferably several or more preferably of a plurality of reference curves.

This will be described in more detail below. The reference curves are in each case analyzed beforehand for the large variety of known sample materials by means of the same arrangement 100 and in particular under the same conditions and by means of the same temperature of heat flow program as well as the same measuring points as during the analysis of the actual sample 1, and the results are stored beforehand as reference curves for \dot{Q}(T) or \dot{Q}(t) or dT/dt (t) or dT/dt (T) in a database, which the data processing and control system 60 can access.

The reference curves can hereby in each case be measured beforehand for a known, uniform sample material or a known sample material mixture with two or more defined, known components or for an expected, undesired component, for instance a possible contamination and can be stored in the database.

The reference profiles available in the database preferably comprise at least one selection of pure substances and mixtures, for example plastics and plastic mixtures, which is adapted to the respective application. The database can be provided on a suitable storage medium, not illustrated in the figures, or can be accessible via a network from a remote storage medium.

Based on the result of the comparison, the at least one component of the sample material is, or several components of the sample material are identified in the sixth step S6.

It is further illustrated in FIG. 7 in a dashed line that the steps S3a, S4a or S3b, S4b, respectively, can be carried out repeatedly. It can thus be provided, for example, to carry out several heating and/or cooling processes by means of the temperature-controllable plate-shaped elements 3 and 4, and to repeatedly heat up and cool down the sample material or the at least one component, respectively, in order to repeatedly melt or freeze the sample material or the component(s), respectively-in the case of a phase transition-or to allow it to soften or solidify-in the case of a glass transition. In the case of conversions in the material, which occur due to the temperature change over time, additional endothermic or exothermic changes, glass transitions or phase transitions can be detected and used for the identification in this way, depending on the sample material or component(s) thereof. For example, the step S5 of comparing can hereby be carried out immediately after detecting the profile, which can be represented as curve, as outlined in an exemplary manner in FIG. 7, or, alternatively, all provided heat-up/cool-down processes can initially be carried out, the obtained curve profiles can be stored and, collectively, the comparison can then be carried out in step S5. It can additionally be provided hereby that the obtained profiles for all heat-up/cool-down processes are not mandatorily considered for the comparison but that a defined selection is provided. It can in particular be advantageous to use the second heat-up for the comparison because said heat-up makes it possible to assume a uniform initial state.

The evaluation in step S5 takes place by applying the heat flow \dot{Q}(T) over temperature or \dot{Q}(t) over time or by applying the temperature change dT/dt (t) over time or dT/dt (T) over temperature.

In step S5, the comparing of the curve state of the curve detected for the sample 1 and applied in the above-mentioned manner, to the reference curve state in particular also comprises a comparison of the curve shape of the curve applied for the sample 1 and of the reference curve(s).

Conversions within the sample material manifest themselves by means of levels or extrema in these curve profiles. The comparison of curve state and reference curve state can be carried out by using corresponding abscissa and/or ordinate values, which in each case characterizes the curve shape and the reference curve shape at least in sections. By determining, for example, onset, endset and/or midpoint temperatures at the curve measured and applied for the sample 1 for the heat flow \dot{Q}(t), \dot{Q}(T) or the temperature change dT/dt (t), dT/dt (T) or of abscissa values, respectively, for instance temperatures, at which extrema occur, and a conclusion can additionally be drawn to the sample compositions, for example on the basis of the integral surfaces under peaks. For this purpose, in particular the characteristic temperatures are compared as characteristic abscissa values, in particular the mentioned onset, endset, midpoint and extreme temperatures, optionally the integral values and/or the curve profiles in their entirety, to the reference curves in the database as well as corresponding characteristic temperatures determined beforehand for these reference curves. The ordinate values, which are assigned to the above-mentioned characteristic abscissa values, can also be compared. The database includes reference curves of pure substances and mixtures, which are relevant for the application.

As part of the evaluation in step S5, it can, for example, further be provided in the case of some exemplary embodiments of the method that a height of the level is determined for at least one level of the obtained profile of the heat flow or of the temporal change of the temperature and is compared to a height of at least one level of the reference curve(s). For example, an extreme value of the curves of heat flow or temporal temperature change itself as ordinate value can analogously be compared directly to an ordinate value of an extreme of the reference curve(s).

A characteristic integral value in terms of a surface under at least one section of the curve for heat flow or temporal change of the temperature, which was obtained for the analysis of the sample 1, can additionally be calculated during the evaluation in step S5 and can be compared to integral values of reference curves, in order to improve the evaluation even further.

Exemplary embodiments are further possible, in the case of which the obtained profile of the heat flow or of the temporal change of the temperature is compared directly to the reference profile in each case in the totality thereof in step S5. The curve shape and the reference curve state are hereby preferably compared to one another by using a pattern recognition algorithm, for example an adaptive algorithm and/or an artificial intelligence method.

In the case of some exemplary embodiments, it can additionally be provided in step S5 to “fit” the result curve, in other words, the curve obtained for the heat flow or the temporal change of the temperature, in each case applied over time or temperature, with several reference curves when a material mixture is present in the sample 1, i.e., to carry out a fitting of several reference curves to the actual measuring result, and to draw a conclusion to the composition of the sample material of the sample 1 from the resulting weights of the individual reference curves. For example, a characteristic peak of each of the curves can be possible for a fitting of this type, whereby the fitting then focusses, for example, on a curve section with such a peak.

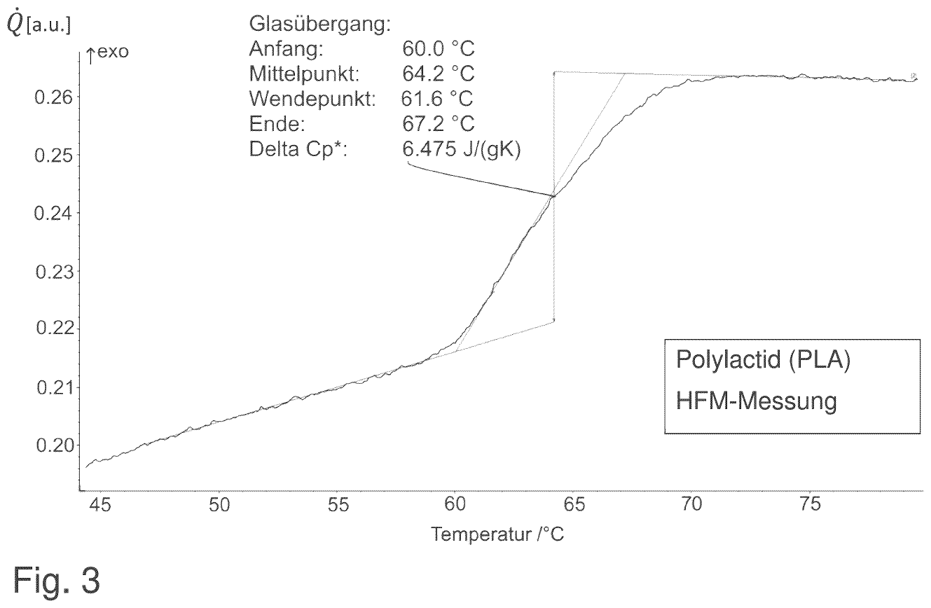

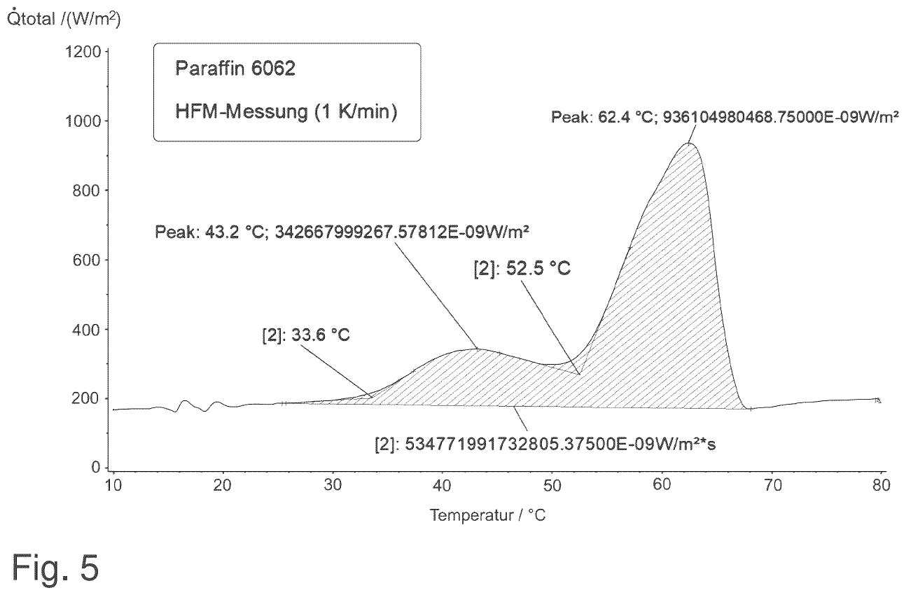

Heat flow curves, which were obtained for an impressed temperature change in the form of a linear ramp with the HFM-like arrangement 100 according to FIG. 1 according to exemplary embodiments of the invention and which are thus referred to as “HFM measurement”, are illustrated in FIG. 3 for a polylactide (PLA) and in FIG. 5 for a paraffin, here, for example, paraffin 6062. Some characteristic values identifying the curve shape in the respectively illustrated section, which were each derived from \dot{Q}(T) curve, are specified in FIGS. 3, 5.

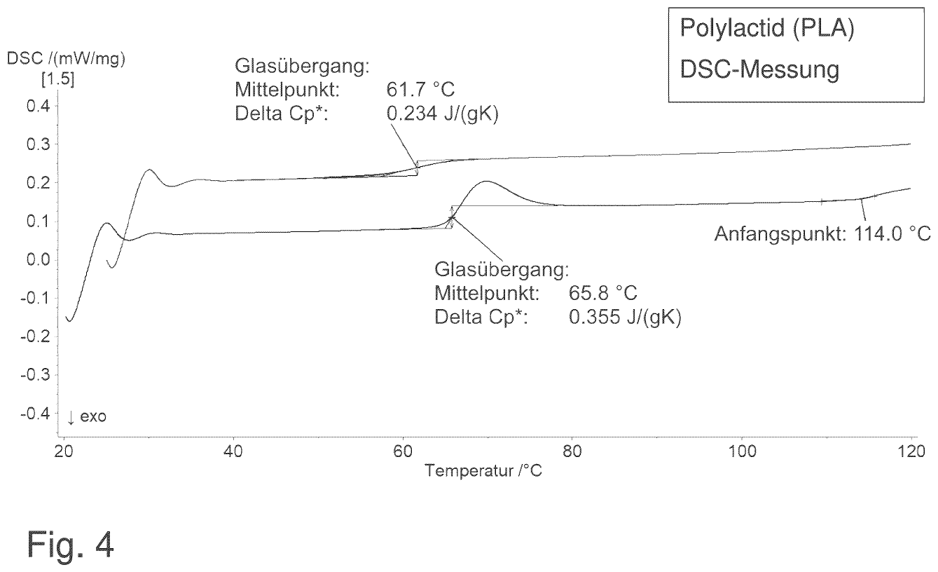

FIG. 3 illustrates, for example, that a glass transition to PLA appears during the “HFM measurement” in the second heat-up as a level at approx. 65 degrees Celsius, with a midpoint of the level at 64.2° C. In FIG. 3, the heat flow Q is applied in an arbitrary unit (abbreviated with a.u.) for illustrating in particular the curve shape and the characterization thereof. In comparison, FIG. 4 shows the result of a conventional DSC measurement (differential scanning calorimetry) for two heat-ups of the same polylactide, but on a significantly smaller sample mass. In the case of the example shown in FIG. 4, a midpoint of the level of 65.8° C., which corresponds to the glass transition, is obtained during the first heat-up and 61.7° C. during the second heat-up, in each case in the conventional DSC method.

The comparison of the result obtained according to an exemplary embodiment of the invention in FIG. 3 for the second heat-up to the DSC results in FIG. 4 for the first and second heat-up generally shows a good similarity with regard to the curve shape and the numerical values. The method proposed by the invention thus makes it possible to achieve results in the manner of a DSC. Reference curves for known sample materials for developing a database, as described further above, for the identification and/or classification of the components of a sample material to be analyzed are preferably created in the HFM-like method of the invention, for instance by means of the arrangement 100, in order to achieve the best possible comparability.

It can be advantageous in many cases to use the second heat-up of the sample material, such as, for instance, for PLA in FIG. 3, in the method according to exemplary embodiments of the invention because the second heat-up assumes a better-defined initial state.

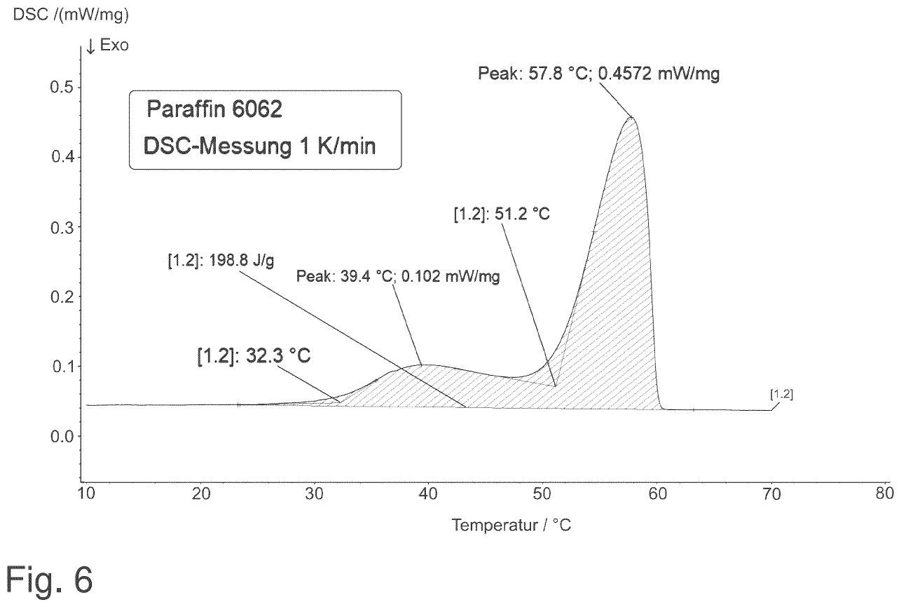

For the evaluation, which is shown in an exemplary manner and which is carried out by means of a method according to an exemplary embodiment, of the heat flow profile, which was measured in the case of impressed continuous temperature change in the form of a linear ramp with a gradient of 1 Kelvin per minute by means of an arrangement 100 according to FIG. 1 for the paraffin 6062 during first heat-up, an integral value is represented under a section of the obtained curve in a shaded manner in FIG. 5 in addition to the abscissa and ordinate values of two peaks and further temperature values characterizing the curve shape. FIG. 5 shows a melt transition of the paraffin 6062. It is important to mention that in the case of the paraffin 6062, the second heat-up provides very similar results. In comparison, the result of a conventional DSC measurement for the first heat-up of the same paraffin is represented, in turn, in FIG. 6, which, in turn, shows the good conformity with the result in FIG. 5 obtained according to an exemplary embodiment of the invention, in particular also with regard to the curve shape.

If, for example, the different paraffins 6062 and 5254 according to yet a further exemplary embodiment are analyzed and the measuring result is evaluated, the melting thereof presents itself as appearance of maxima in a temperature range between 50° C. and 70° C.

The method, as it is described above on the basis of exemplary embodiments, can be used, for example, in the purity determination of a plastics recyclate, for example of a granulate made from recycled plastic. The method according to the above-explained exemplary embodiments hereby offers a simple, time-and cost-saving as well as exact and reliable option for determining whether the granulate contains admixtures or contamination from the recycling process, in particular from other plastics, and to what extent. The above exemplary embodiments, which introduce heat in a symmetrical manner into the sample 1 with the plate-or disk-like geometry 2 through main surfaces 21, 22 of the latter or remove it from said sample, respectively, provides for the analysis of batches at an industrially relevant scale, such as during recycling processes, due to the relatively large possible weighted samples of sample material. The effort for the analysis of the batch is manageable and feasible here.

The analysis of the batch by analyzing the sample 1 can be used easily, quickly and efficiently, for example, in the granulate-producing operation for the quality control of the outgoing batch or analogously at the customer, for instance a plastics processor, for the incoming inspection and can provide reliable, exact results. The sample material of the sample 1 can be plastic, which has already been compounded.

It is advantageous in the case of the invention that a measurement on a reference material or an empty measurement as reference, simultaneously with the measurements on the actual analysis object, thus the sample 1, is not necessary, as it is typically provided, for instance, in the case of the conventional DSC method by means of a second, empty crucible. The arrangement 100 of FIG. 1, as it is used for the exemplary embodiments described here, does not include any units for simultaneously carrying out a reference measurement. An empty “twin” of the arrangement 100 as reference is also not required for carrying out the method according to the exemplary embodiments of the invention described here. The effort for carrying out the method according to the exemplary embodiments described here is thus limited in an advantageous manner in terms of apparatuses as well as in terms of the handling.

Depending on the application and the sample materials to be analyzed, the arrangement 100 can be formed analogously to an HFM apparatus, which is generally known as such, such as, for instance, the one described in ATSM C 1784, and can be modified according to the temperatures, at which the exothermic/endothermic processes, which are to be observed, are expected in the sample material. An HFM apparatus, for example for carrying out the method described in the above exemplary embodiments, can be modified hereby for higher and/or lower temperatures and/or a wider temperature range, which the apparatus is to make possible and for which it is to be suitable, for example, in terms of the performance, layout or dimensioning of the heating and cooling units 7, 8, 9, 10, of possible sealing materials and optionally of further constituents. For the application in the field of plastics recycling, the arrangement 100 is thus formed, for example, in such a way that phase and/or glass transitions of the relevant, expected plastics as main and contamination constituents of the sample material are reliably captured by the temperature range, which the arrangement 100 makes possible.

Even though the invention has been described completely above on the basis of preferred exemplary embodiments, it is not limited thereto but can be modified in a variety of ways.

1. A method for identifying at least one component of a sample material, with which a sample is formed, comprising:
providing the sample, whereby the sample material is formed with a plate-or disk-like geometry or the sample material is arranged in a spatial region with a plate-or disk-like geometry;
arranging the sample between two temperature-controllable plate-shaped elements in such a way that the temperature-controllable plate-shaped elements extend along a respective one of two opposite main surfaces of the plate-or disk-like geometry;
bringing about a temperature change of the sample by bringing about a heat inflow into the sample or a heat outflow out of the sample in a symmetrical manner through the two main surfaces of the plate-or disk-like geometry, whereby the temperature change is effected by means of temperature-controlling the plate-shaped elements and covers a temperature range, within which at least one endothermic or exothermic change of the at least one component of the sample material occurs;
detecting a heat flow flowing into the sample or out of the sample and obtaining from this a profile of the heat flow, which can be represented as curve, as a function of a temperature of the sample or of the temperature-controllable plate-shaped elements or as a function of time, or
detecting a temperature of the sample or of the temperature-controllable plate-shaped elements, and obtaining from this a profile of a temporal change of the temperature, which can be represented as curve, as a function of temperature or as a function of time;
comparing a curve state, in particular including a curve shape, of the obtained profile of the heat flow or of the temporal change of the temperature over temperature or time in each case to a reference curve state of one or several reference profiles; and
identifying the at least one component of the sample material based on the result of the comparison.

2. The method according to claim 1, wherein the sample material is subjected to a continuous temperature profile, in particular a predefined temperature ramp, preferably a temperature ramp, which rises or falls linearly over time, when bringing about the temperature change of the sample.

3. The method according to claim 1, wherein the bringing about of the temperature change of the sample takes place in such a way that a heat flow, which is constant over time, is supplied to the sample or is removed from the sample.

4. The method according to claim 1, wherein the at least one component of the sample material runs through a phase transition and/or a glass transition within the temperature range covered during the temperature change.

5. The method according to claim 1, wherein the sample material is melted or frozen completely or partly during the temperature change.

6. The method according to claim 1, wherein the sample material is subjected to two or more temperature change processes, in particular to one or several heating processes and one or several cooling processes, by means of temperature-controlling the temperature-controllable plate-shaped elements.

7. The method according to claim 1, wherein a heat flow, which in each case flows from the temperature-controllable plate-shaped element into the sample or vice versa, is in each case detected between the temperature-controllable plate-shaped element and the side of the sample facing the temperature-controllable plate-shaped element in the region of the main surface of the plate-or disk-like geometry, to which the temperature-controllable plate-shaped element is adjacent, in particular in each case by means of a heat flux transducer, for example of a heat flux transducer, which is formed in a planar manner.

8. The method according to claim 7, wherein the heat flux transducers are in each case arranged in a planar manner in a central region of one of the main surfaces of the plate-or disk-like geometry between the temperature-controllable plate-shaped element adjacent to this main surface and the sample.

9. The method according to claim 1, wherein one or both of the temperature-controllable plate-shaped elements is/are moved towards the sample prior to bringing about the temperature change of the sample, whereby, conveyed via the temperature-controllable plate-shaped elements, a predefined force is applied to the sample in a thickness direction of the plate-or disk-like geometry or a predefined thickness of the sample is adjusted in the thickness direction of the plate-or disk-like geometry.

10. The method according to claim 1, wherein the temperature of the sample is detected on a surface of the sample or in an interior of the sample and/or that the temperature of the temperature-controllable plate-shaped element is in each case detected on a surface thereof or in an interior thereof.

11. The method according to claim 1, wherein the comparing of the curve state and of the reference curve state is carried out by using corresponding abscissa and/or ordinate values, which in each case characterizes the curve shape and the reference curve shape at least in sections.

12. The method according to claim 1, wherein the comparing of the curve state and of the reference curve state is carried out by means of at least one characteristic level and/or of at least one characteristic extremum, in particular peak, in each case of the obtained profile of the heat flow or of the temporal change of the temperature as well as of the reference profile, whereby the level or the extremum in each case corresponds in particular to a conversion within the sample material.

13. The method according to claim 1, wherein at least one abscissa value, which corresponds to an onset point or endpoint or midpoint or turning point or extreme value assigned to the level or the extremum within the curve shape, is determined for at least one level or at least one extremum, in particular a peak, of the obtained profile of the heat flow or the temporal change of the temperature, and is compared to at least one corresponding abscissa value of at least one reference profile.

14. The method according to claim 1, wherein a height of the level is determined for at least one level of the obtained profile of the heat flow or of the temporal change of the temperature, and is compared to at least one height of at least one level of at least one reference profile, and/or that the assigned extreme value of the heat flow or of the temporal change of the temperature is determined for at least one extremum, in particular a peak, of the obtained profile of the heat flow or of the temporal change of the temperature, and is compared to an extreme value of at least one extremum, in particular peak, of at least one refence profile.

15. The method according to claim 1, wherein at least one characteristic integral value, in particular a surface below at least one section of the obtained profile of the heat flow represented as curve or of the temporal change of the temperature, for example under a peak, is determined and compared to a characteristic integral value of the reference profile and that the identification of the at least one component of the sample material is additionally carried out based on the result of the comparison of the characteristic integral values.

16. The method according to claim 1, wherein the obtained profile of the heat flow or of the temporal change of the temperature is compared directly to the reference profile in each case in the totality thereof.

17. The method according to claim 1, wherein the comparing of the curve state, in particular curve shape, and of the reference curve state, in particular reference curve shape, includes the use of a pattern recognition algorithm, for example of an adaptive algorithm and/or of an artificial intelligence method.

18. The method according to claim 1, wherein a plurality of profiles of the heat flow or of the temporal change of the temperature, which are in each case determined beforehand for a known sample material or sample material mixture, are used as reference profiles.

19. The method according to claim 1, wherein the reference profiles are provided in the form of a data collection, which includes reference profiles of an application-specific selection of pure substances and mixtures, for example plastics and plastic mixtures.

20. The method according to claim 1, wherein the sample material is provided as a loose material with a plurality of individual pieces, for example as a granulate or a powder or that the sample material is provided as a cohesive body.

21. The method according to claim 1, wherein the detection of the profile of the heat flow or of the temperature takes place in the absence of a simultaneous reference measurement, in particular on a reference, which geometrically corresponds to the sample and which is formed with a previously known material, or by means of empty measurement as reference, within the same analysis arrangement or by means of a further identical analysis arrangement.

22. The method according to claim 1, wherein the analyzed sample has a mass of more than approximately 10 grams, for example between approximately 10 grams and approximately 10 kilograms, preferably between approximately 10 grams and approximately 1 kilogram, more preferably between approximately 10 grams and approximately 200 grams, for example between approximately 10 grams and approximately 100 grams.

23. The method according to claim 1, wherein the sample material is a mixture and that a material or several materials, which is/are contained in the sample, is/are identified by means of the method.

24. The method according to claim 1, wherein the method is used for determining the purity of a plastics recyclate, in particular for identifying possible admixtures or contaminations and/or for determining an extent thereof.

25. The method according to claim 1, wherein the method is used in a process chain during the plastics recycling.

26. The method according to claim 1, wherein a compounded substance mixture, in particular a compounded plastic, is provided as the sample material of the sample.
